(19) United States
(12) Patent Application Publication
(10) Pub. No.: US 2012/0171186 A1
(43) Pub. Date: Jul. 5, 2012

(54) METHOD FOR PRODUCING A NUTRACEUTICAL COMPOSITION AND THE NUTRACEUTICAL PRODUCED BY THE METHOD

(76) Inventor: Woon San Liang, UEP Subang Jaya, (MY)

(21) Appl. No.: 13/321,569

(22) PCT Filed: Apr. 19, 2010
(86) PCT No.: PCT/MY10/00062

§ 371 Date: Nov. 21, 2011

(30) Foreign Application Priority Data
May 25, 2009 (MY) PI 20092122

(51) Int. Cl. A61K 36/28; A61K 31/122; A61P 17/18; A61P 1/00; A61P 31/12; A61P 31/10; A61P 37/04; A61K 38/43; A61P 31/04

(52) U.S. Cl. 424/94.1; 424/764; 514/690

(57) ABSTRACT

A method for producing a nutraceutical composition comprising a combination of oil soluble antioxidants derived from an edible oil and water soluble antioxidants derived from a plant extract, wherein the plant extract further provides a natural surfactant; and the nutraceutical composition produced by that method.

This invention relates to a method for producing a nutraceutical and the nutraceutical produced by the method. More particularly, this invention relates to a nutraceutical composition comprising a combination of oil soluble antioxidants and water soluble antioxidants and its production method.

### DESCRIPTION OF THE PRIOR ART

Nutraceuticals are generally a combination of food extracts that have a documented physiological benefit on human health. Consumption of nutraceuticals has been claimed to reduce the risk of certain chronic diseases e.g. diabetes, hypertension, immuno-regulatory diseases etc. Nutraceuticals where a majority of the constituents are naturally-derived would obviously be preferred over those having a mostly synthetic content. Of course, 100%-natural nutraceuticals are particularly preferred.

Generally, the major nutrient constituents in naturally-derived nutraceuticals are antioxidants, which are known to be efficacious in the treatment and prevention of a wide range of chronic illnesses. Aquatic animal oil, in particular krill oil or fish oil such as cod liver oil shark liver oil are a rich source of antioxidants such as omega-3 fatty acids and squalene. Examples of phyto-antioxidants found in vegetables and herbs are pigments such as carotenoids and phenolics or more accurately described as polyphenolics such as flavonoids, saponins, tocopherols and tocotrienols. Polyphenol antioxidants are generally believed to be instrumental in combating oxidative stress in humans, a process associated with some neurodegenerative diseases and some cardiovascular diseases.

Flavonoids are well known water soluble antioxidants and have relatively low toxicity in comparison to other phytochemical compounds. Flavonoids may be divided into six subclasses e.g. anthocyanidins, flavanols, flavanones, flavonols, flavones and isoflavones. Examples of common dietary flavonoids are resveratrol (red wine), catechin (tea), epicatechin (cocoa), hesperidin (citrus fruits), genistine and daidzein (soybean) and quercetin (capers).

Saponins are amphipathic glycosides that dissolve in water to form a stable soapy froth. As a natural surfactant, they are often used as emulsifiers and are excellent detergents. Use of saponins in nutraceuticals for controlling cholesterol-related illnesses has been notably successful. Saponin acts to lower blood cholesterol levels by binding cholesterol and preventing its re-absorption into the blood circulatory system, when consumed. Natural saponins have been known to alter permeability of cells (promote absorption of medicine), and are capable of altering the surface tension of water. The antibacterial, antiviral, antifungal and detoxification properties of saponin have also been documented in numerous scientific studies.

Other examples of phytonutrients are trace minerals such as potassium, calcium and magnesium. These minerals are documented to be efficacious in preventing and managing certain mineral-deficiency mitigated disorders.

Carotenoids (vitamin A) are organic pigments naturally occurring in the chromoplasts of plants. Along with tocopherols and tocotrienols (vitamin E), carotenoids are examples of phytonutrients that are natural oil-soluble antioxidants believed to play a significant role in the prevention of cancer, cellular aging and/or treatment of atherosclerosis, arthritis and Alzheimer's disease, and are available in significant amounts in some edible vegetable oils such as red palm oil, wheat germ oil, coconut oil, corn oil, soya bean oil, olive oil, sunflower oil, rice bran oil or grape seed oil.

Typically, nutraceutical compositions are formulated in various forms depending on intended application e.g. solutions, colloidal dispersions, oil-in-water or water-in-oil suspensions, creams, gels, lotions, powders, foams, mousses, suppository etc.

An example of an oil-form phytochemical composition is disclosed in U.S. Pat. No. 6,596,306 B1 (US '306). No water soluble antioxidant source is provided in US '306, which essentially discloses a self-emulsifying fat soluble drug having a combination of oil soluble antioxidants (tocotrienols, tocopherols, vitamins A, D and K and β-carotene), an oil (palm olein and soybean oil) and a synthetic surfactant system.

A further example of a phytochemical composition with no water soluble antioxidant source provided is disclosed in U.S. Pat. No. 6,562,372 B1 (US '372). The tocotrienol containing powder of US '372 mainly comprises an oil containing tocotrienol, cellulose, lecithin, an emulsifying agent (gelatin, casein sodium, arabic gum or modified starch), and a powder substance as an oil absorbent.

This invention thus aims to alleviate at least some of the problems of the prior art and to provide a method of producing a nutraceutical composition having a combination of water soluble and oil soluble antioxidants and the nutraceutical produced by said method.

### SUMMARY OF INVENTION

In accordance with an aspect of the invention, there is provided a method for producing a nutraceutical composition comprising a combination of oil soluble antioxidants and water soluble antioxidants derived from a plant extract, wherein the plant extract further provides a natural surfactant. The method includes the steps of:
(i) preparing a plant extract;
(ii) concentrating the plant extract to about 2 to 10% solid content;
(iii) mixing the plant extract concentrate with an edible oil at a predetermined ratio; and
(iv) forming an emulsion by homogenizing the mixture of step (iii).

In an embodiment of the invention, the method further comprises step (v) wherein the emulsion of step (iv) is dried to form a powder composition.

In a further embodiment, the edible oil may be mixed with the plant extract in step (iii) at a ratio in the range of 9:1 to 1:9. The preferred edible oil-plant extract mix ratio is dependent on the intended use of the resultant emulsion of step (iv). For example, a mix ratio of 1:9 is preferred if the emulsion is to be administered by way of an enema and a mix ratio of 1:1 is preferred for oral administration forms.

According to another embodiment, a binding agent is added to the resultant mixture of step (iii), for example, maltodextrose. The amount of binding agent added to the mixture may be up to 50% of the total edible oil-plant extract mixture content and may generally be determined by the mix ratio of the edible oil and plant extract.

For example, when the mixture of step (iii) has a ratio of 1:1, then the binding agent may be added to the edible oil-plant extract mixture at a preferred ratio of 1:1:1.

The binding agent may be added to the edible oil-plant extract mixture of step (iii) at a ratio of 1:9:2.

The binding agent may be added to the edible oil plant extract mixture of step (iii) at a ratio of 9:1:5.

According to a further embodiment, the plant extract may comprise flavonoids, surfactants such as saponin, carbohydrates and trace minerals such as magnesium and potassium.

The plant extract may be derived from the Vernonia amygdalina plant.

The extract may be prepared from the leaf, stem, root or a combination thereof, of the Vernonia amygdalina plant.

The extract may be prepared from the leaf of the Vernonia amygdalina plant.

Fresh green Vernonia amygdalina leaves may be used to prepare the plant extract. In this embodiment, step (i) includes:
(a) producing a paste from fresh Vernonia amygdalina leaves;
(b) diluting the paste of step (a);
(c) removing large fibrous articles from the diluted mixture of step (b);
(d) adding diatomaceous earth powder to the liquid supernatant step (c);
(e) filtering the mixture of step (d) to obtain Vernonia amygdalina extract.

Step (a) may comprise blending, grinding or using a screw-press juicer to produce said paste.

The dilution of step (b) may comprise addition of water at a ratio of 4 parts water to 1 part of Vernonia amygdalina leaves used for production of the paste.

Diatomaceous earth powder may be added to the liquid of step (c) at a ratio of from about 5 to about 10% of diatomaceous powder to about 95 to about 90% liquid.

Separation of non-water soluble solids from water soluble solids of the liquid mixture of step (d) with a decanter apparatus may be performed, prior to step (e).

The filtration of step (e) may comprise a ceramic tube filtration process by compressed air.

Pressed palm fiber may be added to the liquid extract of step (e) and further processed in a mixer to form an emulsion. Solid and fiber content are subsequently removed to form a concentrated liquid emulsion.

When fresh Vernonia amygdalina leaves are used for preparation of the plant extract, a freeze drying method is preferably used in step (v) to produce a dried powder composition.

Dried Vernonia amygdalina leaves may also be used to prepare the plant extract. In this embodiment, step (i) includes:
(a) adding water to dry Vernonia amygdalina leaves at a predetermined ratio and soaking the mixture for an amount of time;
(b) heating the mixture of step (a) for an amount of time;
(c) allowing the heated mixture of step (b) to cool to room temperature; and
(d) filtering the cooled mixture of step (c) to obtain Vernonia amygdalina extract.

Water may be added to the dry leaves in step (a) at a ratio of about 1 part leaf to 8 parts water.

The water and dry leaf mixture may be soaked for about 1 to 2 hours in step (a).

The mixture of step (a) may be heated to a temperature below boiling temperature, in step (b). Preferably, the mixture is heated to a temperature between about 50° C. to about 80° C. The heating of the mixture in step (b) may be conducted for about 2 to 3 hours.

The resultant plant extract from dried Vernonia amygdalina leaves may preferably be concentrated to about 5% solid content in step (ii).

When dried Vernonia amygdalina leaves is used for preparation of the plant extract, a spray drying method is preferably used in step (v) to produce a dried powder composition.

According to another embodiment, the edible oil may comprise one or more nutrients selected from a group comprising omega-3 fatty acids and squalene. Examples of such edible oil are aquatic animal oil such as krill oil and edible fish oil. Examples of fish oil having the above nutrients are cod liver oil or shark liver oil.

In another embodiment, the edible oil is an edible vegetable oil that comprises one or more phytonutrients selected from a group comprising tocopherols, and tocotrienols. Examples of such vegetable oil include red palm oil and pressed palm fiber oil.

The edible oil may also comprise phytonutrients such as tocopherols, tocotrienols, carotenoids, phytosterols, squalene and co-enzyme Q-10. Examples of such vegetable oil include red palm oil, wheat germ oil, coconut oil, corn oil, soya bean oil, olive oil, sunflower oil, rice bran oil or grape seed oil.

Red palm oil is preferably used.

In a yet further embodiment, the emulsion of step (iv) is further processed to produce a form suitable for administration by way of an enema.

In a further embodiment, the dried powder composition of step (v) may be further processed to produce nutraceuticals suitable for oral administration. The oral administrative form may comprise tablets or capsules.

In another embodiment, the dried powder composition of step (v) may be further processed to produce nutraceuticals suitable for topical administration. The topical administrative form may comprise topical creams or gels.

The dried powder composition of step (v) may also be mixed with juices, alcoholic beverages and powdered beverages to produce enriched forms of those beverages. For example, the dried powder may be mixed with fruit juices (guava juice), beer, powdered form of chocolate and malt milk beverage as well as coffee powder.

According to another embodiment, the dried powder composition of step (v) may be mixed with liquid deodorized cocoa butter. The dried powder of step (v) may be mixed with deodorized cocoa butter at a ratio of about 5 to 50% dried powder to about 95 to 50% cocoa butter.

The resultant mixture may be further processed to form nutraceuticals suitable to for oral administration. The oral administrative form may comprise chewable capsules.

The resultant mixture may be further processed to form nutraceuticals suitable for colorectal administration. The colorectal administrative form may comprise a suppository.

According to another aspect of the invention, a nutraceutical composition comprises a combination of oil soluble antioxidants and water soluble antioxidants derived from a plant extract, wherein the plant extract further provides a natural surfactant to the composition.

In an embodiment of this aspect, the plant extract may be derived from the Vernonia amygdalina plant.

According to another embodiment of this aspect, the oil soluble antioxidant may be derived from krill oil or edible fish oil such as cod liver oil or shark liver oil.

In a further embodiment of this aspect, the edible oil may be edible vegetable oil such as red palm oil, pressed palm fiber oil, wheat germ oil, coconut oil, corn oil, soya bean oil, olive oil, sunflower oil, rice bran oil or grape seed oil.

The nutraceutical composition of this invention may be used in the manufacture of a medicament for the prevention of human health complications which may comprise cancers of the breast, lung, prostate or colon, cardiovascular disease, fatty liver, asthma, rheumatoid arthritis, dermal allergies, edema, diabetes, hypertension and hyperglycemia.

According to an embodiment, the composition may be used as an antioxidant, anti-bacterial, anti-viral or anti-fungal agent.

According to another embodiment, the composition may be used as an immunostimulator.

According to a further embodiment, the composition may be used as a detoxification agent including that suitable for colorectal cleansing.

Water soluble components are generally known to be more quickly and easily absorbed by the human body in comparison with oil soluble components. However, certain nutrients are either only or preferably deliverable in oil soluble form. The above applies equally to plant derived or animal derived antioxidants.

Hence, it would be advantageous to produce a nutraceutical composition having a combination of water soluble antioxidants and oil soluble antioxidants. In order to successfully combine these naturally immiscible components, it would be necessary to include a surfactant in the composition.

It is an object of this invention to provide a method of producing a nutraceutical composition comprising a naturally derived surfactant in addition to a combination of water soluble and oil soluble antioxidants as well as the nutraceutical produced by said method.

It is particularly preferred that the nutraceutical composition produced by the method of this invention be 100%-natural i.e. comprising of only naturally derived components.

Edible vegetable oils (e.g. red palm oil, pressed palm fiber oil, wheat germ oil, coconut oil, corn oil, soya bean oil, olive oil, sunflower oil, rice bran oil or grape seed oil) as well as aquatic animal oil (e.g. krill oil or edible fish oil such as cod liver oil or shark liver oil) are a rich source of oil soluble antioxidants. It would generally be preferable for these edible oils to be delivered with a natural emulsifier in a method of this invention.

Plants comprising natural surfactants such as saponins as well as significant amounts of water soluble antioxidants would be particularly suitable for use in the method of this invention.

It was surprisingly found that the Vernonia amygdalina plant (common name: bitterleaf, ewuro, ndole and onugbu) contained a significant amount of a natural surfactant (e.g. saponins) in addition to water soluble antioxidants (e.g. quercetin equivalent flavonoids) and beneficial trace minerals (magnesium and potassium). This herbal shrub from the family Asteraceae originates from Nigeria, Africa. It is a well known medicinal plant and has been traditionally used for the treatment of malaria fever, headache, joint pain associated with AIDS, diabetes (U.S. Pat. No. 6,531,461 B2), stomach illnesses as well as cancer prevention (U.S. Pat. No. 6,849,604 B2). It can be eaten as a leafy vegetable. The roots have been used in the treatment of gingivitis and toothache due to its proven antimicrobial activity.

The method of this invention advantageously allows for production of a nutraceutical comprising a combination of oil and water soluble antioxidants as well as a naturally derived surfactant that may be delivered by a variety of routes, for example, oral, topical, colorectal.

The nutraceutical produced from the method of this invention may also be advantageously stored in a variety of forms i.e. liquid, powder, gel etc.

Further, the nutraceutical produced may also be advantageously used for the prevention or treatment of a variety of human health complications.

The above-described advantages of the method of the present invention therefore, provide for a method that successfully enables production of a nutraceutical comprising a combination of oil soluble and water soluble antioxidants as well as a naturally derived surfactant.

### BRIEF DESCRIPTION OF THE DRAWINGS

The invention is illustrated, although not limited, by the following description of embodiments made with reference to the accompanying drawings in which:

FIGS. 1A and 1B are pictures of the Vernonia amygdalina plant, source of the water soluble antioxidant and natural surfactant in a preferred embodiment of the invention.

FIG. 2 is a picture of fresh leaves of the Vernonia amygdalina plant used as a source of the plant extract in the preferred embodiment of the invention.

FIG. 3 are HPLC chromatograms of a fresh sample of Vernonia amygdalina leaves at 200 nm, 254 nm and 366 nm.

FIG. 4 are HPLC chromatograms of a freeze-dried (soaking) sample of Vernonia amygdalina leaves at 200 nm, 254 nm and 366 nm.

FIG. 5 are HPLC chromatograms of a spray-dried (60° C.) sample of Vernonia amygdalina leaves at 200 nm, 254 nm and 366 nm.

FIG. 6 is a comparison of the HPLC chromatograms of FIGS. 3 to 5.

FIG. 7 is a UV spectrum of the major peaks observed in the HPLC chromatograms of FIGS. 3 to 5.

### DEFINITIONS

Unless otherwise defined the following terms as used throughout this specification are defined as follows: —

“Antioxidant” as used herein refers to a molecule capable of slowing or preventing the oxidation of other molecules.

“Binding agent” as used herein refers to a compound that binds together various components for improving yield during production of a powder form.

“Emulsion” as used herein refers to a mixture of two immiscible liquids, a water soluble liquid and an oil soluble liquid.

“Flavonoids” as used herein refers to a class of secondary plant metabolites having a three-ringed flavone backbone, known for their antioxidant activity.

“Nutraceutical” as used herein refers to a composition comprising a combination of food extracts claimed to have a physiological benefit on human health and/or reduce the risk of chronic disease in humans.

“Phenols” or “phenolics” as used herein refers to chemical compounds consisting of a hydroxyl group (—OH) bonded directly to an aromatic hydrocarbon group.

“Phytochemical” or “phytonutrient” as used herein refers to plant-derived chemical compounds under scientific research for their potential health promoting properties.

“Polyphenols” as used herein refers to a group of chemical substances characterized by the presence of more than one phenol unit or building block per molecule.

“Saponin” as used herein refers to amphipathic glycosides composed of one or more hydrophilic glycoside moieties combined with a lipophilic triterpene derivative.

### DETAILED DESCRIPTION OF THE EMBODIMENTS

The method for producing a nutraceutical composition comprising a combination of oil soluble antioxidants derived from an edible oil and water soluble antioxidants derived from a plant extract, the plant extract further providing a natural surfactant, mainly includes the steps of preparing a plant extract, concentrating the extract to about 2 to 10% solid content, mixing the extract concentrate with an edible oil at a predetermined ratio and forming an emulsion by homogenizing the mixture.

### The Plant Extract Component

Generally any plant comprising a significant amount of natural surfactant and water soluble antioxidants may be used in the method of this invention. Plants from the Asteraceae family such as Vernonia spp. or the Acanthaceae family such as Strobilanthes crispus or Saricocalix crispus may be used.

In a preferred embodiment, the herbal plant, Vernonia spp. was used. Various species of Vernonia spp. may be used e.g. Vernonia amygdalina, Vernonia colorata, Vernonia calvoana etc. It is particularly preferred to use Vernonia amygdalina.

Any part of the Vernonia amygdalina plant (e.g. root, stem, leaf or a combination thereof) may be used for preparation of the extract for production of the nutraceutical composition of this invention. However, it is particularly preferred that the leaves of the Vernonia amygdalina plant be used due to its higher phytonutrient content and generally more palatable taste in comparison to other parts of the plant. Either fresh green leaves or dry leaves of the plant may be used for preparation of the Vernonia amygdalina extract with fresh leaves being preferred over dry leaves. This is because the Vernonia amygdalina extract produced from fresh leaves of the plant was found to contain less impurities than that produced from dry leaves.

Preparation of the Vernonia amygdalina Extract

Methods generally known in the art may be used for preparation of the plant extract from its root and stem. Hence, only the preparation method of the Vernonia amygdalina extract from the plant leaves will be discussed herein.

From fresh green leaves of the Vernonia amygdalina plant

The preparation of a liquid extract from fresh green leaves of the Vernonia amygdalina plant in a preferred embodiment of the invention is described below.

Firstly, the fresh green Vernonia amygdalina leaves are rinsed with filtered water. The rinsed leaves are then cut into smaller sizes to facilitate preparation of a Vernonia amygdalina leaf paste. The paste may be prepared by either blending or grinding the cut leaves. Alternatively, a screw press juicer or any other commercial device suitable to produce the leaf paste may also be used. Water is then added to the Vernonia amygdalina leaf paste at a ratio of about 4:1 (e.g. 4 kg of water to every 1 kg of fresh green leaves used) to produce a first liquid extract.

Large fibrous particles are sieved out of the first liquid extract to form a second liquid extract.

About 5 to 10% of food grade diatomaceous earth powder (DE powder) is added to the second liquid Vernonia amygdalina extract and the mixture is stirred for about 10 to 15 minutes. The DE powder facilitates removal of chlorophyll from the mixture.

The mixture then undergoes a purification step via a ceramic tube filtration process by compressed air. The liquid mixture is filled into a filtration vessel containing multiple ceramic filter tubes sequentially arranged. The ceramic filter tubes used herein are devoid of filtration agents (e.g. activated carbon). Clean filtered compressed air is then pumped through the filtration vessel to initiate the filtration process. Residual non water soluble solids (DE powder) are removed with this filtration process.

Optionally, the second liquid extract-DE powder mixture may also be flowed through a decanter apparatus for separation of non water soluble solids (e.g. DE powder and fine fibre particles) and water soluble solids, prior to the above filtration process.

The final liquid Vernonia amygdalina extract used for preparation of the nutraceutical composition of this invention is honey brown in colour and contains only water soluble solids.
From dried leaves of the Vernonia amygdalina plant

A preferred method of preparing the liquid extract from dried Vernonia amygdalina leaves will now be described below.

Water is added to dry Vernonia amygdalina leaves at a ratio of preferably about 8:1 (e.g. 8 kg water to 1 kg leaves). The leaves are left to soak for about 1 to 2 hours. The soaked leaves are then heated to a temperature of about 50° C. to about 80° C. (any temperature above 50° C. but below boiling temperature) for about 2 to 3 hours to produce a first liquid extract.

The first liquid extract is allowed to cool to room temperature before being filtered to obtain a final (second) liquid Vernonia amygdalina extract which is dark brown in colour and may be used for preparation of the nutraceutical composition of this invention.

### The Edible Oil Component

Edible vegetable oil

Various edible vegetable oil may be used in combination with the above final liquid Vernonia amygdalina extract for production of the nutraceutical composition of this invention.

Usage of edible vegetable oil comprising oil soluble antioxidants such as tocopherols and tocotrienols is preferred. Examples of vegetable oil having such phytonutrient constituents are red palm oil, wheat germ oil, coconut oil, corn oil, soya bean oil, olive oil, sunflower oil, rice bran oil or grape seed oil.

Vegetable oil such as red palm oil or pressed palm fiber oil having carotenoids, phytosterols, squalene and co-enzyme Q-10, in addition to tocopherols and tocotrienols, are particularly preferred.
Edible aquatic animal oil

Aquatic animal oil having oil soluble antioxidants such as omega-3 fatty acids and squalene may also be used in combination with the Vernonia amygdalina extract for production of the nutraceutical composition of this invention. Krill oil or edible fish oil such as cod liver oil or shark liver oil, are preferred examples of such edible oil.

The edible oil used, whether vegetable or aquatic animal derived, may be mixed with the final liquid Vernonia amygdalina extract at varied predetermined ratios in the manner as further described below.

### Preparation Method of the Nutraceutical Composition

The prior prepared liquid Vernonia amygdalina extract is concentrated to a concentration of about 2 to 10% solid content by conventional methods.

A concentration of 5% solid content for extract-derived from dry Vernonia amygdalina leaves is preferred.

The Vernonia amygdalina extract concentrate is then mixed with the edible oil (e.g. red palm oil or cod liver oil etc.) in a homogenizer. Any conventional equipment/apparatus that allows for complete homogenization of the extract-edible oil mixture may be used in the method of this invention.

The extract may be mixed with the edible oil at various predetermined ratios e.g. 1:9 (10% Vernonia amygdalina extract water soluble solid content) or 1:4 (20% Vernonia amygdalina extract water soluble solid content) or 1:1 (50% Vernonia amygdalina extract water soluble solid content), or at any mix ratio in the range of 1:9 to 9:1.

The preferred mix ratio of the Vernonia amygdalina extract and oil component is largely dependent on the nutraceutical to be produced. For example a mix ratio of 1 part Vernonia amygdalina extract and 1 part oil component is preferred if a capsule or tablet is to be produced whereas a mix ratio of 9 parts Vernonia amygdalina extract and 1 part oil component is preferred if the nutraceutical is to be administered as an enema.

The Vernonia amygdalina extract contains a natural emulsifier component. Hence, the edible oil is emulsified to form a “plant extract-oil emulsion” post-homogenization.

In an embodiment, the Vernonia amygdalina extract produced from fresh leaves may be used as a “pressed palm fiber oil extractor” when mixed with pressed palm fiber.

Pressed palm fiber oil is the residual oil remaining in palm fibers after extraction of crude palm oil using a screw press; It is impossible to completely extract all the palm oil from the palm mesocarp using mechanical extraction. After extraction of crude palm oil from the mesocarp, the de-oiled mesocarp is know as palm fibers and there is about 5 to 6% oil remaining in these fibers. This residual oil is conventionally extracted either by way of solvent extraction or super critical fluid extraction. The palm fiber oil contains very high levels of carotenoids (4000 to 6000 mg/kg), tocols (2400 to 3500 mg/kg) as well as other phytonutrients such as squalene (1000 to 1800 mg/kg), sterols and phospholipids (about 3.7%).

Fresh clean palm fiber collected from an oil mill is mixed and blended with the liquid extract in a conventional mixer. The saponin (surfactant) content in the liquid extract will act as a “natural detergent” and cause oil to be extracted from the pressed palm fiber when the mixture is squeezed and pressed during the mixing process. As the palm fiber has already been subject to high temperature and high pressure in the oil mill, the oil and water soluble phenolics attached to the palm fiber may be washed out (“squeezed out”) of the palm fiber to form an oil-in-water emulsion with the saponin. As more of the palm fiber is added to the mixture, a portion of the liquid emulsion formed is adsorbed by the fiber which is pressed to form a more concentrated emulsion. When the mixer is stopped, the fiber is squeezed a final time before the extracted liquid emulsion is sieved to remove solids and fiber content. A concentrated liquid extract emulsion comprising Vernonia amygdalina extract and pressed palm fiber oil is produced.

In a preferred embodiment, the emulsion may be converted, via a conventional drying process, to a powder form for storing. Examples of drying methods that may be used are the spray drying or freeze drying method. The freeze drying method is preferred when the plant extract component of the emulsion is derived from fresh Vernonia amygdalina leaves whereas the spray drying method is preferred when dried leaves are used.

### The Nutraceutical Composition

As explained above, the nutraceutical composition of this invention (whether in emulsion or powder storage form) comprises a combination of water soluble antioxidants derived from the Vernonia amygdalina extract and oil soluble antioxidants derived from edible oil (e.g. vegetable or fish oil). Importantly, the nutraceutical composition of this invention can be said to be devoid of synthetic components i.e. 100% natural.

In order to enhance the nutrient content of the nutraceutical composition produced, edible oils having high content of oil soluble antioxidants are preferred for use in the method of this invention.

Edible vegetable oil having antioxidants such as tocopherols and tocotrienols (vitamin E family), carotenoids (vitamin A family), phytosterols, squalene and Co-enzyme Q10 are preferred.

Aquatic animal oil having antioxidants such as omega-3 fatty acids and squalene is preferred. Particularly preferred, are krill oil or edible fish oil.

As later shown in the Examples, the Vernonia amygdalina extract mainly comprises a significant amount of water soluble phytochemicals such as flavonoids (antioxidant), saponin, and trace minerals (e.g. magnesium and potassium).

Flavonoids are well known antioxidants and have been strongly indicated to modify the human body's reaction to allergens and viruses. They are also considered to have some efficacy in the prevention of certain cancers and heart disease.

After analysis, it was found that the flavonoids contained in the Vernonia amygdalina extract were quercetin-equivalents. Quercetin has been found to be the most active flavonoid. It demonstrates significant anti-inflammatory activity, is a potent antioxidant, lowers blood pressure in hypertensive patients and has exhibited some efficacy in the treatment and prevention of cancers, heart disease, cataracts, and respiratory diseases such as bronchitis and asthma.

Saponin, a major constituent of the Vernonia amygdalina extract, is a naturally occurring surfactant i.e. a natural emulsifier. It is the availability of saponin in the extract that advantageously allows for the combination of water soluble antioxidants (flavonoids) with oil soluble antioxidants (e.g. tocopherols, tocotrienols, carotenoids, omega-3 fatty acids, squalene etc.) in the nutraceutical composition of this invention.

Currently, natural saponins derived from the tea plant and plants in the Sapindaceae (e.g. lychee), Quillajaceae, (e.g. soapbark) and Agavaceae (e.g. yuccas) families as well as chemically synthesized surfactants (e.g. lecithin) are generally used as emulsifiers in relation to processing of oil-based formulations or mixtures.

As a surfactant, saponin can be termed as a “natural cleaning agent”, thus, the later description of usage of the nutraceutical composition of this invention as an oral cleansing agent when administered orally and as a detoxification agent when administered via the colorectal route.

Saponin also has antioxidative properties (an antioxidant) that protects cells from oxidative free radicals and is widely known to have anti-bacterial, anti-viral and anti-fungal properties.

Typical of amphipathic (hydrophilic and lipophilic) compounds, soap-like foam is generally observed on the surface of liquids containing saponin, when shaken.

The trace mineral elements (e.g. magnesium and potassium) of the Vernonia amygdalina extract may be effective in alleviating dehydration and in the treatment of illnesses due to particular mineral deficiencies (e.g. asthma).

### Various Formulations

The nutraceutical composition produced by the method of this invention may be further formulated into either liquid or powder forms that may be administered via various routes (e.g. oral, topical/dermal or colorectal route) to a human body.
Liquid form

The liquid emulsion nutraceutical composition of the method of this invention, either by itself or when mixed with other naturally derived food ingredients, can be further processed (e.g. pasteurized) for consumption as health beverages. It is preferred that fresh Vernonia amygdalina leaves are used to prepare the plant extract component of the nutraceutical emulsion when use as a health beverage is intended.

Also, the liquid emulsion may be administered by way of an enema. In this instance, preferably, the emulsion comprises 9 parts plant extract with 1 part edible oil. Either conventional or advanced enema methods may be used.
Powder form

In order to produce a powder form of the nutraceutical, a binding agent is added to the edible oil-plant extract mixture prior to formation of the nutraceutical emulsion by homogenization. The amount of binding agent added to the mixture can be up to 50% of the total edible oil-plant extract mixture content and is generally determined by the mix ratio of the edible oil and plant extract. For example, the binding agent may be added to the edible oil-plant extract mixture at a ratio of 1 part oil:9 part plant extract:2 part binding agent. Or, when the oil:plant extract mix ratio is 9:1, the binding agent may be added at a ratio of 9 part oil:1 part plant extract:5 part binding agent.

Examples of binding agents that may be added are maltodextrose, fossil shell flour etc. Maltodextrose is a preferred binding agent and may be formulated with the edible oil-plant extract mix at a preferred mix ratio of 1:1:1.

Following addition of the binding agent and formation of the nutraceutical emulsion, either a spray drying or freeze drying method may be used to produce the dried powder form of the nutraceutical which can then be further processed with pharmaceutically acceptable carriers into oral administrative forms such as tablets or capsules for consumption as health supplements.

Due to the substantial natural carbohydrate content of the plant extract, it is also possible to produce the powder form of the nutraceutical without addition of a binding agent to the plant extract-edible oil emulsion. However, addition of a binding agent significantly improves the yield of dried powder nutraceutical from the drying process (whether spray drying or freeze drying).

The dried powder form of the nutraceutical composition may also be further mixed with fish oil concentrates (e.g. cod fish oil, shark fish oil etc.) and gelatin to produce soft gelatinous capsules. The saponin content of the plant extract in the nutraceutical improves the bioavailability of the fish oil concentrate by aiding in the formation of a bio-adsorbable emulsion as the capsule content contacts the water content of the digestive system.

The powder form of the nutraceutical may also be mixed with various beverages for the production of enriched forms of those beverages e.g. fruit juices such as guava juice, alcoholic beverages such as beer and powdered beverages such as chocolate malt flavoured milk beverages (Milo™) and coffee.

In a further option, the powder nutraceutical composition produced by the method of this invention may also be dispersed in an aqueous solution for production of cosmetic or medicinal ointments/creams.
Formulated with deodorized cocoa butter

Cocoa butter is one of the most stable fats known, containing natural antioxidants that prevent rancidity thus allowing for long shelf-life of up to two to five years. Its smooth texture, sweet fragrance and emollient property make it a popular base ingredient in cosmetics and skin care products, such as soaps and lotions.

The dried powder form of the nutraceutical composition of this invention is mixed with deodorized cocoa butter to produce a further variety of health formulations for human administration as described below.

In room temperature, the cocoa butter base is in solid form and may be “indirectly melted” to produce a liquid cocoa butter base. For example, the cocoa butter may be melted in a double layer container where its upper layer contains solid cocoa butter and bottom layer contains warm water. Any other manner of melting the cocoa butter that does not involve a direct application of heat may also be used.

The dried powder nutraceutical is poured into the melted cocoa butter base at a ratio of about 5 to 50% dried powder to about 95 to 50% cocoa butter and stirred to form a liquid nutraceutical-cocoa butter mixture.

When the mixture is thoroughly liquefied and mixed, the liquid mixture may be poured into suitably sized mouldings to form solid mixtures. The moulds are placed in a freezer for a suitable amount of time until the mixture solidifies (e.g. about 15 to 20 minutes).

The shape and size of the mould used may vary according to the intended usage of the solid mixture. For example, the solid mixture may be further processed to produce oral or colorectal administrative forms.

### (i) Oral Administrative Form

An example of the oral administrative form that may be produced from the solid mixture is chewable tablets.

When placed in the oral cavity, the nutraceutical powder content in the tablets absorbs saliva upon contact and normal human body temperature (normal core temperature of about 37° C.; normal oral cavity temperature of about 36.8° C.) causes the cocoa butter content to “melt”, resulting in dissolving of the tablet. Due to the presence of saponin (the nutraceutical powder content), the dissolved tablet mixture produces a “shampoo in mouth” effect. Gargling with the dissolved mixture for about 10 to 15 minutes may improve oral health as the mixture aids in cleansing and disinfecting of gums and teeth.

This would prove useful and convenient for example for geriatric or bedridden patients, or even for travellers or day-to-day usage in the event of water shortage.

In view of the 100% naturally derived content of the tablet, the dissolved mixture may be swallowed after gargling, for example for easing stomach digestion. The dissolved mixture is easily broken down by the human digestive system, thus allowing for effective absorption of the phytochemical content.

### (ii) Colorectal Administrative Form

An example of the colorectal administrative forms that may be produced from the solid mixture is suppositories.

When a suppository comprising the solid nutraceutical-cocoa butter mixture is inserted into the rectal area, the nutraceutical powder content in the suppository absorbs water upon contact with rectal mucus. The normal human body temperature (about 37° C.) causes the cocoa butter content to “melt”, resulting in dissolving of the suppository. Due to the presence of saponin (the nutraceutical powder content), the dissolved suppository produces a “shampoo” effect that aids in colon cleansing and disinfection of the colorectal area.

At the same time, the presence of colorectal bacterium aids in further breaking down the dissolved suppository mixture thus allowing for efficient absorption of the nutraceutical content through the colorectal mucosal lining, while the non-absorbable cocoa butter base is passed out.

Direct absorption into the blood circulatory system via the colorectal mucosal lining which constitutes a substantially large absorption surface, makes this a particularly efficacious delivery route for the nutraceutical composition. Also, the saponin content in this “nutraceutical-suppository” is believed to be capable of increasing colorectal mucosal permeability thus facilitating and stimulating nutrient and water absorption in the colorectal area. Recent research (2007 study conducted by Linus Pauling Institute) has indicated that flavonoids are in fact poorly absorbed by the human body (less than 5%) and those absorbed are quickly metabolized and excreted. Hence, it is postulated that the role of saponin in the nutraceutical composition of this invention is particularly significant in the delivery efficacy of the antioxidant nutrient (flavonoid) content of the composition.

The water soluble chemical content (flavonoids and trace minerals) of the nutraceutical mixture and cocoa butter provides instant energy to the human body when absorbed through the colorectal mucosal lining. Again, this would be particularly useful for geriatric or bedridden patients that have minimally or non-functioning digestive system or an inability to consume oral nutrients. Such an instant energy source may also prove useful for inhabitants or travelers in extremely cold areas. Bypassing the oral nutrition route, may also prove helpful in weight reduction and in the control of obesity.

As opposed to colorectal administration by way of an enema, suppositories allow for a slow release of the nutraceutical composition i.e. longer nutrient retention time, due to their solid (to semi-solid) nature. Slow release of the nutraceutical composition also reduces the possibility of disruption or distress to the digestive system.

### EXAMPLE

The following Examples illustrate the various aspects, methods and steps of the nutraceutical production method of this invention. These Examples do not limit the invention, the scope of which is set out in the appended claims.

The plant source of the Vernonia amygdalina extract used in the following examples has been planted on 2 acres of land in Assam Jawa, Kuala Selangor, Selangor, Malaysia.

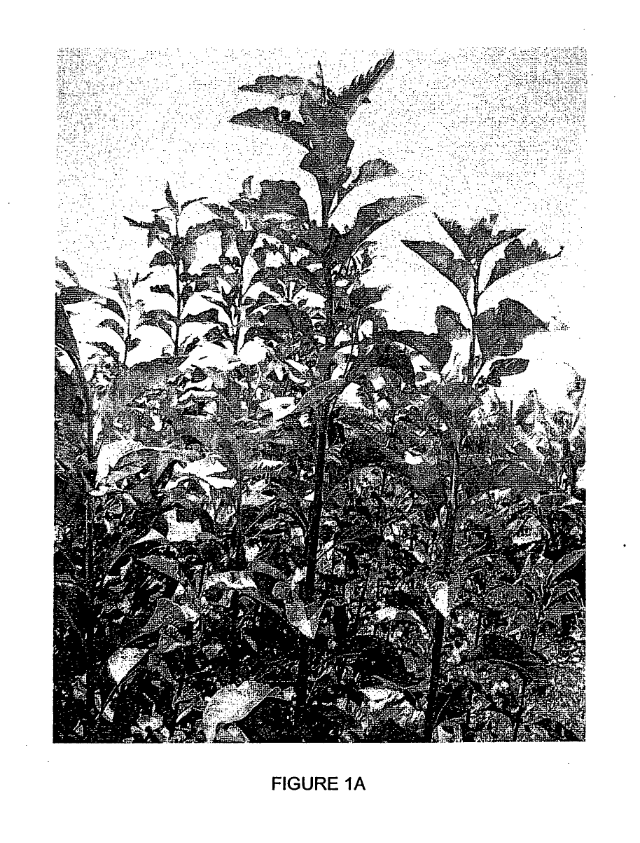

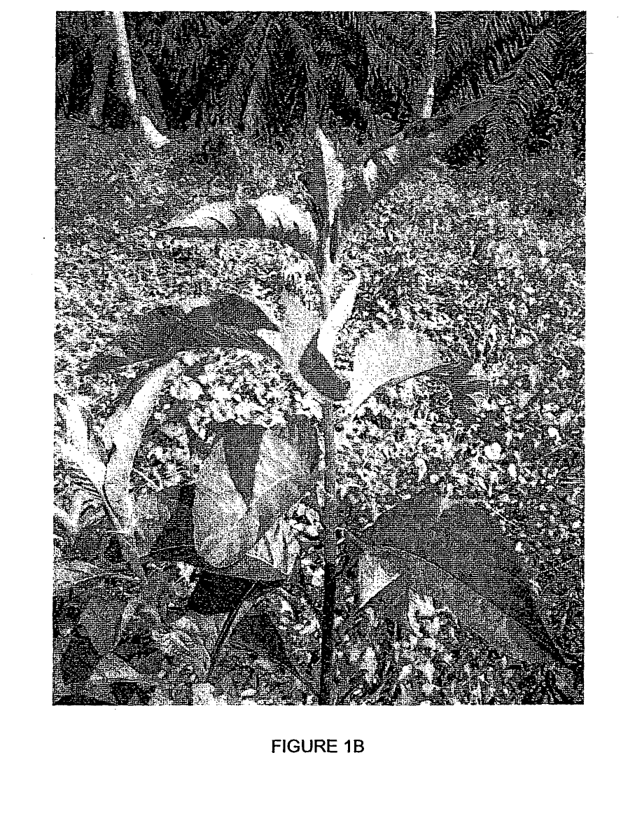

The Medicinal Plants Division of the Forest Research Institute of Malaysia or FRIM has confirmed, by taxonomical identification*, that the plant belongs to the genus Vernonia in the family Asteraceae. From examining the reproductive parts of the plant, FRIM found that it closely resembles Vernonia amygdalina. The Vernonia amygdalina plant is as seen in FIGS. 1 and 2. *FRIM ref. FRIM394/TU671/3/1 KLT dated 29 Feb. 2008

### Example 1

### General Nutritional Value Analysis on the Spray-Dried Powder Form of a Vernonia amygdalina Dry Leaf Extract

Several known test methods were done to ascertain the general nutritional value a Vernonia amygdalina dry leaf extract. This analysis was conducted by Consolidated Laboratory (M) Sdn Bhd*. The results are shown in Table 1 below. *Lab ref. CL/M116116/2007 dated 6 Aug. 2007

TABLE 1
Result of the general nutritional value analysis on a spray-dried
powder form of the Vernonia amygdalina dry leaf extract.

| Test parameter | Unit | Test method | Result |
|---|---|---|---|
| Fat | g/100 g | Soxhlet extraction | ND (<0.1) |
| Carbohydrate | g/100 g | By calculation | 59.9 |
| Protein | g/100 g | Kjeldahl method | 6.7 |
| Dietary fibre | g/100 g | AOAC 985.29 | 1.3 |
| Moisture | g/100 g | Oven method | 9.11 |
| Ash | g/100 g | Furnace method | 24.31 |

As can be observed, the Vernonia amygdalina leaf extract has substantially high natural carbohydrate content.

### Example 2

### Heavy Metals and Minerals Test on the Spray-Dried Powder Form of a Vernonia amygdalina Dry Leaf Extract

An Inductively Coupled Plasma Mass Spectrometry (ICP-MS) detection method was done by the Spectra Kembangan Laboratory of the Faculty of Chemical And Natural Resources Engineering, Universiti Teknologi Malaysia to ascertain the heavy metals and minerals constituents of a Vernonia amygdalina dry leaf extract.

The results are as shown in Table 2.

TABLE 2
Result of the heavy metals and minerals test on a spray-dried
powder form of the Vernonia amygdalina dry leaf extract.

| Heavy Metal/Mineral Constituent | Amount (mg/l) |
|---|---|
| Lead (Pb) | 0.17 |
| Mercury (Hg) | Not Detected |
| Copper (Cu) | 0.52 |
| Zinc (Zn) | 2.06 |
| Arsenic (As) | 0.10 |
| Iron (Fe) | 9.23 |
| Magnesium (Mg) | 282.72 |
| Nickel (Ni) | 0.41 |
| Cadmium (Cd) | 0.01 |
| Chromium (Cr) | 0.28 |
| Cobalt (Co) | 0.01 |
| Calcium (Ca) | 31.61 |
| Sodium (Na) | 32.62 |
| Phosphorus (P) | 0.51 |
| Selenium (Se) | Not Detected |
| Aluminum (Al) | 0.42 |
| Silver (Ag) | 0.01 |
| Barium (Ba) | 0.08 |
| Potassium (K) | 533.89 |

Based on the above analysis result, the Vernonia amygdalina leaf extract contains high trace amounts of potassium, magnesium, calcium and sodium and does not contain toxins or heavy metals.

### Example 3

### Phytochemical Analysis on the Vernonia amygdalina Plant

Bioassays* were performed by the Medicinal Plants Division of FRIM to ascertain the phytochemical constituents (e.g. alkaloids, saponins, flavonoids, tannins, triterpenes and steroids) in a sample of a Vernonia amygdalina leaf extract. *FRIM ref FRIM394/TU671/1/1(Sub5)/S17 dated 9 Sep. 2005

### Alkaloids Assay

At least 2.5 grams of Vernonia amygdalina leaves were macerated in chloroform followed by addition of ammoniacal chloroform. The chloroform was drawn off and filtered into a test tube or the like. A sufficient amount was added into the test tube and the chloroform layer was separated. After removal of the chloroform layer, the liquid extract obtained was treated with 10% sulphuric acid and subsequently tested with Mayers reagent.

Formation of white precipitates is indicative of the presence of alkaloids. No white precipitate was observed i.e. no alkaloids present in the Vernonia amygdalina leaf extract.

### Saponin Assay

A standard methanol froth test was conducted to detect the presence and relative amount of saponin present in the Vernonia amygdalina leaf extract. The liquid leaf extract diluted in methanol was mixed with distilled water in a test tube.

Formation of stable froth for at least 15 minutes is indicative of the presence of saponin. Stable froth of about 2 to 3 cm in height (+2 on standard scale of +1 to +3) was observed i.e. substantial amount of saponin present in the Vernonia amygdalina leaf extract.

### Flavonoid Assay (Flavonoid Presence)

A standard flavonoid detection test was conducted to detect the presence and relative amount of flavonoid present in the Vernonia amygdalina leaf extract. The leaf extract diluted in chloroform was dissolved in ether. The mixture was added to a volumetric flask containing a 10% ammonia solution and shaken.

Formation of yellow color in the ammonia solution is indicative of the presence of flavonoids. A mild yellow color (+2 on standard scale of +1 to +3) was observed i.e. substantial amount of flavonoid present in the Vernonia amygdalina leaf extract.

### Tannins and Polyphenolic Compounds Assay

The Vernonia amygdalina leaf extract diluted in methanol was mixed with 1% ferric chloride solution and shaken.

Formation of blue black color is indicative of the presence of hydrolysable tannins, while a brownish green color is indicative of the presence of condensed tannins. No color change was observed i.e. no tannins present in the Vernonia amygdalina leaf extract.

### Sterols/Titerpenes Assay

The Vernonia amygdalina leaf extract diluted in chloroform was tested using the Liebermann-Buchard reagent.

Formation of reddish color is indicative of the presence of triterpenes and a greenish color is indicative of the presence of sterols. A mild greenish color (+2 on standard scale of +1 to +3) was observed i.e. no triterpenes present in the Vernonia amygdalina leaf extract and substantial amount of sterols present in the Vernonia amygdalina leaf extract.

The results of the above phytochemical analysis on the Vernonia amygdalina leaf extract is summarized in Table 3.

TABLE 3
Summary results of phytochemical analysis of a
sample of Vernonia amygdalina leaf extract.

| Constituent | Amount |
|---|---|
| Alkaloids | Not Detected |
| Saponins | 2+ |
| Flavonoids | 2+ |
| Tannins/Polyphenolics | Not Detected |
| Triterpenes | Not Detected |
| Sterols | 2+ |

1+ Weak; 2+ Mild; 3+ Strong

In summary, the Vernonia amygdalina leaf extract comprises significant amounts of saponins, flavonoids and steroids.

### Example 4

### Saponin Content Analysis of Vernonia amygdalina Leaf Extract

Total triterpenoid saponin analysis of a Vernonia amygdalina leaf extract based on ursolic acid equivalent was performed by the Medicinal Plants Division of FRIM.

A colorimetric method was used for triterpenoid saponin determination with slight modifications. 0.5 or 1.0 ml of diluted leaf extract in methanol was transferred into a 10 ml test tube. Methanol was evaporated by immersing the test tube in a water bath. 0.2 ml of newly mixed 5% (w/v) vanillin-acetic acid solution and 1.2 ml of perchloric acid were added. The mixture was then incubated at 70° C. for 15 minutes. The test tube was taken out and cooled in running water for 2 minutes. Then, ethyl acetate was added in order to make the total volume 5 ml. Absorbance of the mixture was determined with a spectrophotometer at 550 nm. The result is expressed in % ursolic acid w/w of extract by comparison with ursolic acid standard curve.

The total triterpenoid saponin content of the Vernonia amygdalina leaf extract was observed to be 8.08±0.83% (w/w) (ursolic acid equivalent).

### Example 5

### Flavonoid Equivalent Test on a Vernonia amygdalina Leaf Extract

A flavonoid equivalent test was performed by the Medicinal Plants Division of FRIM to ascertain the flavonoid type and amount present in a sample of a Vernonia amygdalina leaf extract.

The type of flavonoid present in the Vernonia amygdalina leaf extract was identified as a quercetin equivalent. The total quercetin equivalent content in a spray-dried form of the Vernonia amygdalina leaf extract was analysed by way of the aluminum chloride colorimetric method with slight modifications (Liu B. et. al., 2007, Journal of Food Engineering 78: 584-587).

1 ml of diluted extract in methanol, 0.3 ml of sodium nitrate, and 4.0 ml of methanol were mixed and left to stand for 5 minutes. 0.3 ml of 10% aluminum chloride (w/v) was added to the mixture and it was left to stand. Six minutes later, 2.0 ml of 1M sodium hydroxide was added. The final reaction mixture volume was made up to 10 ml by adding 2.4 ml of deionized water prior to measurement at 510 nm with a spectrophotometer.

The results are expressed in quercetin weight (mg) per dry weight of sample (g) by comparison with a quercetin standard curve. The results are shown in Table 4 below:—

TABLE 4
Results of the aluminum chloride colorimetric
assay to determine flavonoid amount present in sample
of Vernonia amygdalina leaf extract.

| | |
|---|---|
| Sample | Flavonoid content |

| | | | |
|---|---|---|---|
| 1 | Soaking at room temperature | 76.3 ± 29.6 | mg/g sample |
| | | 7.6 ± 2.9% | (w/w) |
| 2 | Soaking at room temperature - | 25.9 ± 1.8 | mg/g sample |
| | remainder on dryer | 2.6 ± 0.2% | (w/w) |
| 3 | Heating at 60° C. | 114.8 ± 10.4 | mg/g sample |
| | | 11.5 ± 1.0% | (w/w) |
| 4 | Heating at 60° C. - | 102.8 ± 19.5 | mg/g sample |
| | remainder on dryer | 10.3 ± 1.9% | (w/w) |

Values are the mean ± SD of n = 3 repeats

The above analysis* done by FRIM confirmed the flavanoid content in the Vernonia amygdalina leaf extract to be about 10% by weight percentage i.e. a significant amount. *FRIM ref. FRIM394/TU671/1/1(Sub5)S05 dated 4 Mar. 2008

### Example 6

### High-Performance Liquid Chromatography (HPLC) Analysis on the Vernonia amygdalina Leaf Extract

Three samples of Vernonia amygdalina leaf extract were analysed by HPLC, namely, a fresh sample (code: 157/08), a soaking, freeze-dried sample (code: 096/08) and a 60° C., spray-dried sample (code: 098/08), to ascertain the stability of the flavonoid content in the leaf extract under varying conditions i.e. liquid extract, freeze-dried powder form. The HPLC analysis was performed by the Medicinal Plants Division of FRIM.

5 mg of each sample was mixed with 500 μl water and sonicated for 60 minutes at room temperature. Any insoluble residue was removed by centrifugation. The liquid supernatant was filtered through a PVDF filter (pore size 0.45 μm) into a vial and subsequently used for the HPLC analysis. The three samples, as above prepared, were analysed by means of a HPLC system (Waters Delta 600 with 600 controller) with a photodiode array detector (Water 996). A Phenomenex-Luna (5 μm) column was used (4.6 mm i.d.×250 mm) and for elution of the constituents, a gradient of two solvents denoted as A and B was employed. Solvent A was a 0.1% aqueous H₃PO₄, whereas solvent B was acetonitrile. Initial conditions were 85% A and 15% B, with a linear gradient reaching 25% B at t=12 minutes. This was followed by an isocratic elution until t=22 minutes, after which the programme returned to the initial solvent composition at t=25 minutes and maintained for a further 10 minutes. The flow rate used was 1.0 ml/minute and the injection volume was 10 μl. Three injections were performed for each sample. The retention times and UV spectra of the major peaks in all three samples were analysed.

Some common peaks were found present in all three samples. The concentration (peak intensity) of the chemical components represented by these peaks generally differed from sample to sample in that 098/08 (60° C., spray-dried sample)>096/08 (soaking, freeze-dried sample)>157/08 (fresh sample). Peaks B, D and E were attributed to flavonoids and their intensity remained generally constant for all three samples.

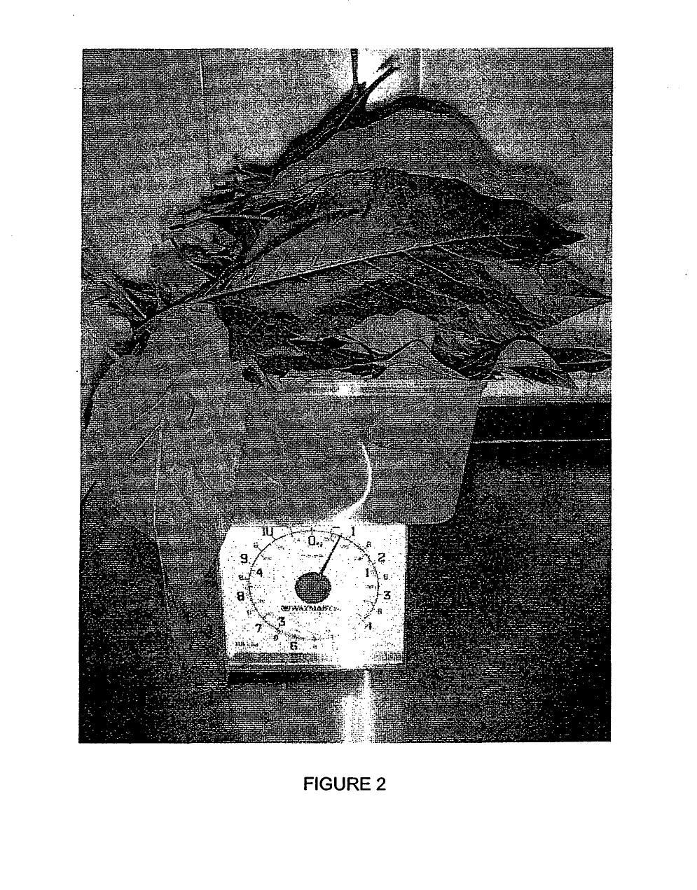

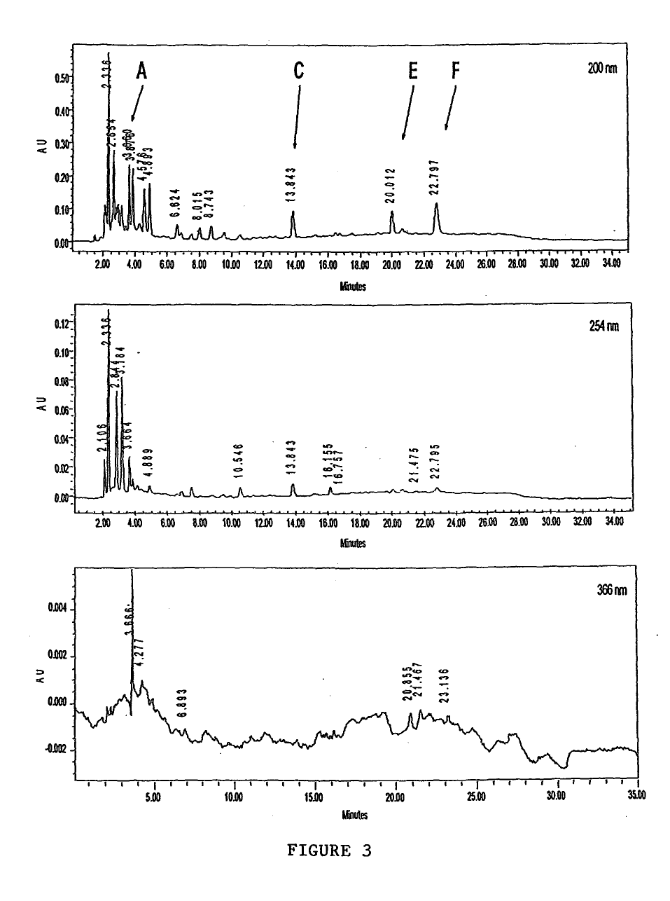

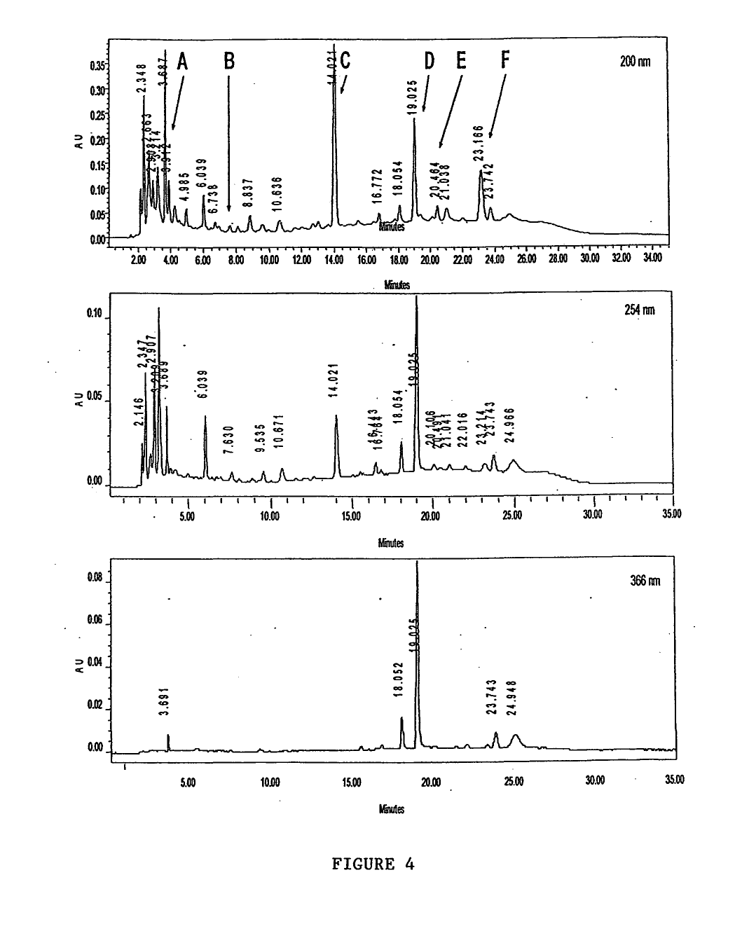

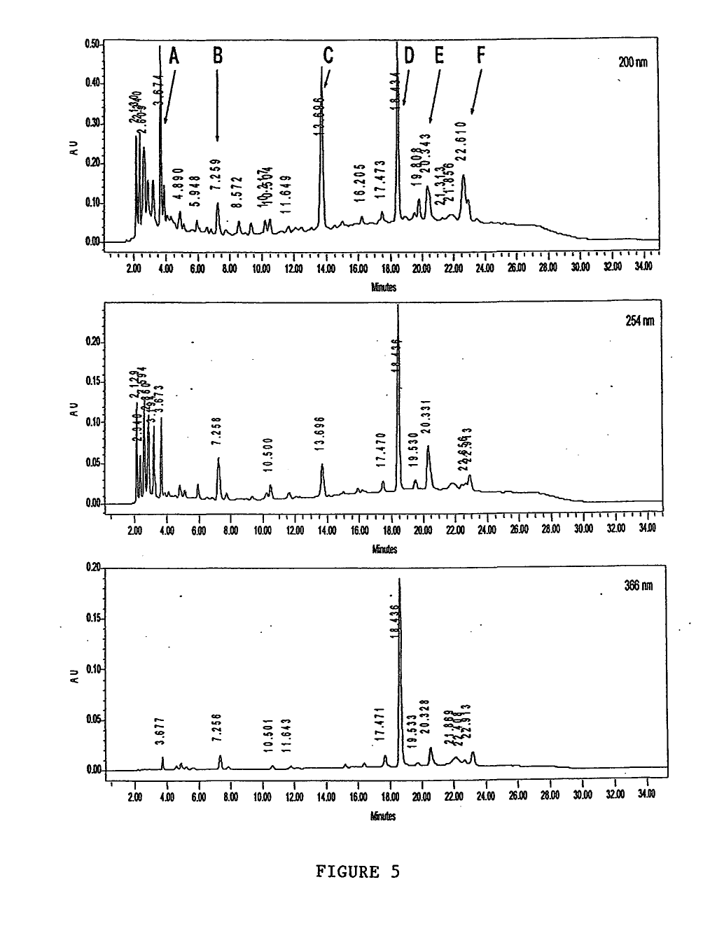

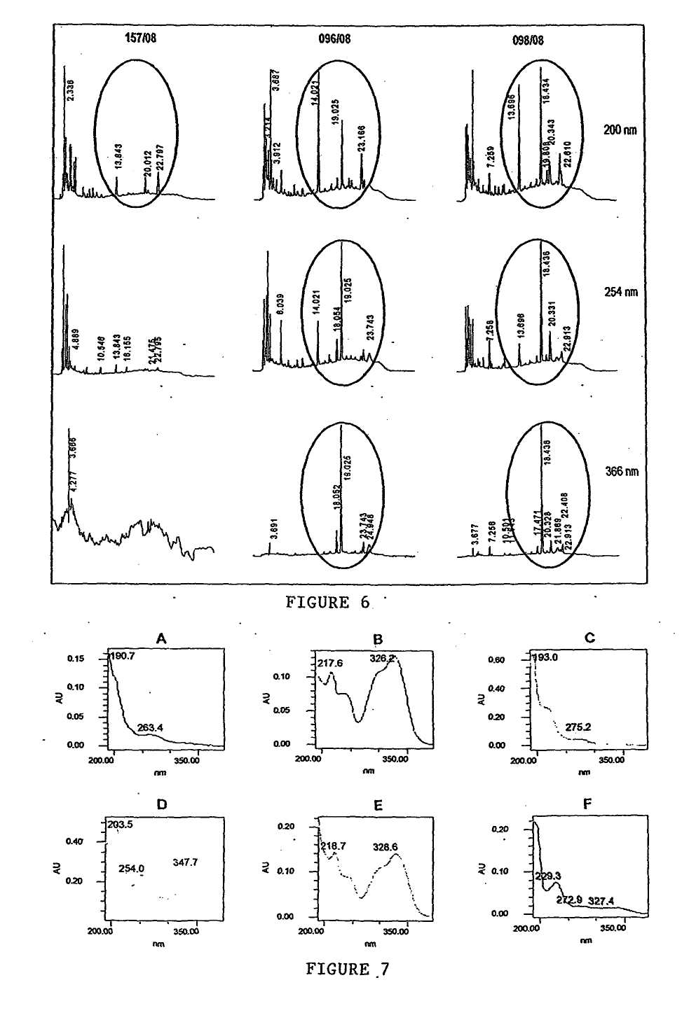

The HPLC chromatograms for all three samples, a comparison of the chromatograms and the UV spectra of major peaks are shown in FIGS. 3 to 7.

### Example 7

### Antioxidant Activity Analysis on the Vernonia amygdalina Leaf Extract

Two assays were performed by the Medicinal Plants Division of FRIM to ascertain the antioxidant activity of a Vernonia amygdalina leaf extract. As seen from the results of Examples 3, 5 and 6 above, the water soluble antioxidant in the Vernonia amygdalina leaf extract has been broadly identified as a type of flavonoid, and more specifically, a quercetin equivalent i.e. the following assays are a test of flavonoid activity.

### Xanthine/Xanthine Oxidase Superoxide Scavenging System

This assay evaluates the scavenging activity of the Vernonia amygdalina leaf extract on any superoxide free radical anions. A standard Xanthine Oxidase reaction mixture was used in this assay.

### DPHP Radical Scavenging Assay

This assay evaluates the reducing activity of the Vernonia amygdalina leaf extract that determines its antioxidant potential (AOP).

Additionally, the total phenolic content (TPC) as well as tyrosinase inhibitory activity of the Vernonia amygdalina leaf extract were also tested. The results of the above test are shown in Table 5 below:—

TABLE 5
Results of the xanthine/xanthine oxidase superoxide scavenging assay, DPHP
radical scavenging assay, total phenolic content and tyrosinase inhibitory
activity tests of a sample of Vernonia amygdalina leaf extract.

| | | Superoxide | DPHP Radical | Total Phenolic | Tyrosinase |
|---|---|---|---|---|---|
| Sample | Sample | Scavenging | Scavenging | Content | Inhibitory |
| Code | description | (%) | (%) | Mg/100 g GAE | Activity (%) |
| Sample | | 0.25 mg/ml | 0.25 mg/ml | 6.25 mg/ml | 2.0 mg/ml |
| Concentration | | | | | |
| Standard | | Superoxide | Ascorbic acid | Gallic acid | L-cysteine |
| | | dismutase | 0.005 mg/ml | (GAE) | (LC) |
| | | (6 × 10⁻³ U/ml) | | standard curve | 0.30 mg/ml |
| S 14/08 | Spray dried | 95.4 H | 60.8 M | 2871.6 H | 55.6 M |
| | leaf extract | | | | |

“UV-VIS Spectrophotometer baseline checked autozero”
H: high; M: moderate; L: low

The results obtained suggest that the Vernonia amygdalina leaf extract shows significant free radical scavenging activity and antioxidant potential as compared with the standard control.

### Example 8

### An Exemplary Protocol for Preparing the Powder Form of the Nutraceutical Composition

The liquid nutraceutical composition used to prepare the spray-dried and freeze-dried powder forms was prepared by way of a method of this invention using Vernonia amygdalina extract derived from dry leaves of the plant and use of red palm oil as the edible oil component.

### Spray Dried Nutraceutical Powder Preparation

The following spray drying process was done by the Chemical Engineering Pilot Plant of Universiti Teknologi Malaysia (Johor) on 11 Nov. 2008.
Batch 1

115 liters of water was added to 12.4 kilograms of dried Vernonia amygdalina leaves. The mixture was heated to a temperature of 60° C. for 3 hours. Heated mixture allowed to cool to room temperature and filtered. 4.5% solid content in a 50 liter (2.25 kilograms) liquid plant extract was observed. 2.25 liter of red palm oil was added to the 50 liter liquid plant extract and homogenized, prior to start of spray-drying process. The inlet temperature of the liquid was 180° C. and outlet temperature was 100° C. The spray drying took 12 hours 30 minutes to complete. Resultant amount of spray-dried powder nutraceutical was 0.523 kg.
Batch 2

115 liters of water was added to 12.3 kilograms of dried Vernonia amygdalina leaves. The mixture was heated to a temperature of 60° C. for 3 hours. Heated mixture allowed to cool to room temperature and filtered. 3.2% solid content in a 70 liter (2.25 kilogram) liquid plant extract was observed. 2.25 liter of red palm oil and 2.25 kilogram of maltodextrose was added to the 70 liter liquid plant extract and homogenized, prior to start of spray-drying process. The inlet temperature of the liquid was 180° C. and outlet temperature was 100° C. The spray drying took 14 hours 30 minutes to complete. Resultant amount of spray-dried nutraceutical powder was 3.696 kg.

In comparison with batch 1, a significantly higher recovery rate of the nutraceutical powder in batch 2 is observed following addition of the binding agent maltodextrose.

### Vacuum Freeze Dried Nutraceutical Powder Preparation

The following freeze drying process was done by the Forest Research Institute of Malaysia (FRIM) on 15 Jan. 2007.

10 kilograms of dried Vernonia amygdalina leaves were soaked in reverse osmosis (RO) water at room temperature for 4 hours. Liquid plant extract subsequently concentrated by way of natural evaporation at 50° C. and mixed with edible oil prior to conventional freeze drying (or lyophilization) technique.

Resultant amount of freeze-dried nutraceutical powder was 1.5 kilograms i.e. 15% yield.

### Example 9

### Oil Soluble Antioxidant Content Analysis on Spray-Dried Powder Form of the Nutraceutical Composition

Tests were performed by the Malaysian Palm Oil Board (MPOB) to ascertain the oil soluble antioxidant content of two samples (batch 1 and batch 2) of spray dried powder nutraceutical composition of this invention as prepared in Example 8 above.

Standard tests were done to ascertain the total carotene content and the presence of various known oil soluble antioxidants in the spray dried powder of batches 1 and 2.

The results of those tests are shown in Table 6 below.

TABLE 6
Results of the oil soluble antioxidant content tests on
two spray-dried samples of the nutraceutical composition.

| | Powder of | | Powder of | | Reference sample | |
|---|---|---|---|---|---|---|
| | Batch 1 | | Batch 2 | | (red palm super olein) | |
| Carotene content | 674 | ppm | 776 | ppm | 581 | ppm |
| Oil content | 18.91% | | 30.46% | | — | |
| Antioxidants | | | | | | |
| Propyl Gallate- | ND | | ND | | ND | |
| PG (ppm) | | | | | | |
| Tert-butyl hydro- | 49.9 | ppm | 11.0 | ppm | ND | |
| quinone-TBHQ | | | | | | |
| (ppm) | | | | | | |
| Butylated hydroxy- | ND | | ND | | ND | |
| anisole-BHA (ppm) | | | | | | |
| Butylated hydroxy- | ND | | ND | | ND | |
| toluene-BHT (ppm) | | | | | | |
| Vitamin E | | | | | | |
| Alpha tocopherol | 426.6 | ppm | 435.9 | ppm | 400.0 | ppm |
| Alpha tocotrienol | 414.4 | ppm | 401.5 | ppm | 435.3 | ppm |
| Gamma tocotrienol | 495.6 | ppm | 476.8 | ppm | 602.6 | ppm |
| Delta tocotrienol | 255.4 | ppm | 247.5 | ppm | 301.7 | ppm |

### Example 10

### Spectophotometric Assay Analysis on the Dried Powder Form of the Nutraceutical Composition

Spectropherometric assays to detect the total phenolic content, total flavonoid content and carotene content were done on the dried powder form of the nutraceutical composition comprising Vernonia amygdalina leaf extract and red palm oil as prepared by the protocol of Example 8. The assays were performed by the Product Development and Advisory Services Division of the Malaysian Palm Oil Board (MPOB).

A Folin Ciocalteu assay was done to determine the total phenolic content of the powder sample. This assay involves measurement of the number of —OH groups present in the sample, assuming that light absorption increases when more —OH groups are present. The Folin-Ciocalteu reagent, a mixture of molybdenum and tungsten salts, was added to the sample. The Mo(VI) and W(VI) are reduced in the presence of the oxidizable phenol groups and a color change was observed. The intensity of the color observed was determined using optical absorption spectroscopy. Quantitation was done by calibration with a gallic acid sample.

A colorimetric assay using NaNO₂ and AlCl₃ (F—Al procedure) was done to determine the total flavonoid content in the powder sample. The spectrophotometric assay based on aluminium chloride complex formation is one of the most commonly used analytical procedures applied to flavonoid content determination in plants. This procedure involved hydrolysis of glycosides, extraction of total flavonoid aglycones with ethyl acetate and complex formation with AlCl₃. A color change was observed and the color intensity was determined using optical absorption spectroscopy. Quantitation was done by calibration with a catechin sample and the results reported as catechin equivalents.

MPOB's in house test method was used to determine the total carotene content in the powder sample.

The results of the above assays are summarized in Table 7 below:

TABLE 7
Summary results of the assays done to determine the total phenolic
content, total flavonoid content and total carotene content
of a dried powder form of the nutraceutical composition.

| Type of Constituent | Amount |
|---|---|
| Total phenolic content | 23.1 mg/g of product acid equivalent |
| (expressed as gallic acid | |
| equivalent) | |
| Total flavonoid content | 15.7 mg/g of product |
| (expressed as catechin equivalent) | |
| Total carotene content | 108 mg/kg |

Note:
Total phenolic content of lettuce is between 18.4 to 52 μg/g and wheat is 2.12 to 3.37 μg/g (gallic acid equivalent).
Total flavonoid content of wheat is between 259 to 330 μg/g of the bran (epicatechin equivalent).
Carotene content of unbleached palm oil is 500 to 700 mg/kg.

As is known, the FCR reagent does not only measure total phenols and generally reacts with any reducing substance. Thus, the above result may in fact be representative of the total reducing capacity (total antioxidant content) of the sample, and not just its total phenolic content.

### Example 11

### Antioxidant Activity Analysis on the Spray-Dried Powder Form of the Nutraceutical Composition

Two assays were performed by the Medicinal Plants Division of FRIM to ascertain the antioxidant activity of two spray-dried powder samples of the nutraceutical composition as prepared in Example 8 above.

### Xanthine/Xanthine Oxidase Superoxide Scavenging System

This assay evaluates the scavenging activity of the spray dried powder samples on any superoxide free radical anions. A standard Xanthine Oxidase reaction mixture was used in this assay.

### DPHP Radical Scavenging Assay

This assay evaluates the reducing activity of the spray dried powder samples that determine their antioxidant potential (AOP).

Additionally, the total phenolic content (TPC) of both spray dried powder samples was also tested.

The results of the above test are shown in Table 8 below:—

TABLE 8
Results of the xanthine/xanthine oxidase superoxide scavenging assay,
DPHP radical scavenging assay and total phenolic content test of
two spray dried powder samples of the nutraceutical composition.

| | | | | Total Phenolic |
|---|---|---|---|---|
| | Sample | Superoxide | DPHP Radical | Content |
| Sample Code | description | Scavenging (%) | Scavenging (%) | Mg/100 g GAE |
| Sample | | 250 μg/ml | 250 μg/ml | 5 mg/ml |
| Concentration | | | | |
| Standard | | Superoxide | Ascorbic acid | Gallic acid |
| | | dismutase | 0.005 mg/ml | (GAE) |
| | | (6 × 10⁻³ U/ml) | | standard curve |
| AO0980309 | Batch 1 powder | 81.5 ± 0.5 H | 56.8 ± 0.25 M | 2717.45 ± 0.92 |
| AO0990309 | Batch 2 powder | 73.8 ± 0.25 H | 19.4 ± 0.55 L | 2497.61 ± 1.79 |

“UV-VIS Spectrophotometer baseline checked autozero”
H: high; M: moderate; L: low
Note:
1. TPC Value > 1000 mg GAE/100 g is considered High Total Phenolic content
2. TPC—Expressed as milligram equivalent to Gallic Acid per 100 g of dry weight (mg GAE/100 g)

### CONCLUSION

The results of Examples 1 to 7 suggest that the Vernonia amygdalina leaf extract contains a substantial amount of starch, various phytochemicals such as saponins, flavonoids, and trace minerals. The flavonoid content alone was about 10% by weight of the dried powder form of the leaf extract. It is evident, from Examples 9 to 11, that the nutraceutical powder contains a combination of Vernonia amygdalina derived water soluble antioxidants and oil soluble antioxidants. Particularly, the nutraceutical powder was observed to have a high phenolic content and a substantial amount of flavonoids, tocotrienols, tocopherols as well as carotene. Hence, it is concluded that the nutraceutical produced by the method of this invention is most suitable for the prevention and/or treatment of various health complications including:

1) Anti bacterial agent
2) Anti viral agent
3) Anti fungal agent

### 4) Antioxidant

### 5) Anti-inflammatory

6) Anti-hypertensive and hyperglycemia agent

### 7) Immunostimulator

8) Amelioration of skin allergy, asthma, and lung respiration complications
9) Detoxification and cleansing agent (colorectal)
10) Water absorption promoter/stimulator (colorectal)
11) Prevention of colon, prostate and lung cancer
12) Improves blood circulation and reduction of cardiovascular disease

As will be readily apparent to those skilled in the art, the present invention may easily be produced in other specific forms without departing from its scope or essential characteristics. The present embodiments are, therefore, to be considered as merely illustrative and not restrictive, the scope of the invention being indicated by the claims rather than the foregoing description, and all changes which come within therefore intended to be embraced therein.

1. A method for producing a nutraceutical composition comprising a combination of oil soluble antioxidants derived from an edible oil and water soluble antioxidants derived from a plant extract, wherein said plant extract provides a natural surfactant to the composition, said method including the steps of:
(i) preparing a plant extract;
(ii) concentrating said plant extract to about 2 to 10% solid content;
(iii) mixing said plant extract concentrate with an edible oil at a predetermined ratio; and
(iv) forming an emulsion by homogenizing said mixture of step (iii).

2. The method of claim 1 further comprising step (v) wherein said emulsion of step (iv) is dried to form a powder composition.

3. The method of claim 1 or 2 wherein said edible oil is mixed with said plant extract in step (iii) at a ratio in the range of 9:1 to 1:9.

4. The method of claim 3 wherein said edible oil is mixed with said plant extract in step (iii) at a ratio of 1:9.

5. The method of claim 3 wherein said edible oil is mixed with said plant extract in step (iii) at a ratio of 1:1.

6. The method of any one of the preceding claims wherein a binding agent is added to the resultant mixture of step (iii).

7. The method of claim 6 wherein said binding agent is maltodextrose.

8. The method of claim 6 or 7 wherein said edible oil, plant extract and binding agent are mixed in step (iii) at a ratio of 1:1:1.

9. The method of claim 6 or 7 wherein said edible oil, plant extract and binding agent are mixed in step (iii) at a ratio of 1:9:2.

10. The method of claim 6 or 7 wherein said edible oil, plant extract and binding agent are mixed in step (iii) at a ratio of 9:1:5.

11. The method of any one of the preceding claims wherein said plant extract comprises flavonoids, surfactants such as saponin, carbohydrates and trace minerals such as magnesium and potassium.

12. The method of any one of the preceding claims wherein the plant extract is derived from the Vernonia amygdalina plant.

13. The method of claim 12 wherein said Vernonia amygdalina extract is prepared from the leaf, stem, root or a combination thereof, of the Vernonia amygdalina plant.

14. The method of claim 12 wherein said Vernonia amygdalina extract is prepared from the leaf of the Vernonia amygdalina plant.

15. The method of claim 14 wherein fresh green Vernonia amygdalina leaves are used to prepare the plant extract.

16. The method of claim 15 wherein step (i) includes:
(a) producing a paste from fresh Vernonia amygdalina leaves;
(b) diluting said paste of step (a);
(c) removing large fibrous articles from said diluted mixture of step (b);
(d) adding diatomaceous earth powder to the liquid supernatant of step (c); and
(e) filtering said mixture of step (d) to obtain Vernonia amygdalina extract.

17. The method of claim 16 wherein step (a) comprises blending, grinding or using a screw-press juicer to produce said paste.

18. The method of claim 16 or 17 wherein the dilution of step (b) comprises addition of water at a ratio of 4 parts water to 1 part of Vernonia amygdalina leaves used for production of said paste.

19. The method of any one of claims 16 to 18 wherein diatomaceous earth powder is added to the liquid supernatant of step (c) at a ratio of from about 5 to about 10% of diatomaceous powder to about 95 to about 90% liquid.

20. The method of any one of claims 16 to 19 wherein a further step of separating non-water soluble solids from water soluble solids of the liquid mixture of step (d) with a decanter apparatus is performed, prior step (e).

21. The method of any one of claims 16 to 20 wherein said filtration of step (e) comprises a ceramic tube filtration process by compressed air.

22. The method of any one of claims 16 to 21 wherein pressed palm fiber is added to the liquid extract of step (e) and further processed in a mixer to form an emulsion.

23. The method of claim 22 wherein solid and fiber content is removed from said emulsion to form a concentrated liquid emulsion.

24. The method of any one of claims 15 to 23 wherein a freeze drying method is used in step (v) to produce a dried powder composition.

25. The method of claim 14 wherein dried Vernonia amygdalina leaves are used to prepare the plant extract.

26. The method of claim 25 wherein step (i) includes:
(a) adding water to dry Vernonia amygdalina leaves at a predetermined ratio and soaking said mixture for an amount of time;
(b) heating said mixture of step (a) for an amount of time;
(c) allowing said heated mixture of step (b) to cool to room temperature; and
(d) filtering the cooled mixture of step (c) to obtain Vernonia amygdalina extract.

27. The method of claim 26 wherein water is added to the dry leaf in step (a) at a ratio of about 1 part leaf to 8 parts water.

28. The method of claim 26 or 27 wherein said water and dry leaf mixture was soaked for about 1 to 2 hours in step (a).

29. The method of any one of claims 26 to 28 wherein said mixture of step (a) was heated to a temperature below boiling temperature in step (b).

30. The method of claim 29 wherein said mixture of step (a) was heated to a temperature of between about 50° C. to about 80° C. in step (b).

31. The method of any of claims 26 to 30 wherein the heating of step (b) was conducted for about 2 to 3 hours.

32. The method of any one of claims 26 to 31 wherein said plant extract is concentrated to about 5% solid content in step (ii).

33. The method of any one of claims 26 to 32 wherein a spray drying method is used in step (v) to produce a dried powder composition.

34. The method of any one of the preceding claims wherein said edible oil comprises one or more nutrients selected from a group comprising omega-3 fatty acids and squalene.

35. The method of claim 34 wherein said edible oil used in step (iii) comprises aquatic animal oil.

36. The method of claim 34 wherein said edible oil used in step (iii) comprises krill oil or fish oil such as cod liver oil or shark liver oil.

37. The method of any one of claims 1 to 34 wherein said edible oil is an edible vegetable oil that comprises one or more phytonutrients selected from a group comprising tocopherols and tocotrienols.

38. The method of claim 37 wherein said edible vegetable oil comprises phytonutrients such as tocopherols, tocotrienols, carotenoids, phytosterols, squalene and Co-enzyme Q10.

39. The method of claim 37 wherein said edible oil used in step (iii) comprises any one of red palm oil, wheat germ oil, coconut oil, corn oil, soya bean oil, olive oil, sunflower oil, rice bran oil or grape seed oil.

40. The method of claim 36 wherein said edible oil used in step (iii) comprises any one of red palm oil or pressed palm fiber oil.

41. The method of any one of claim 37 or 38 wherein said edible oil used in step (iii) comprises red palm oil.

42. The method of any one of claims 1, 4, 11 to 23, 25 to 32 or 34 to 41, wherein said resultant emulsion of step (iv) is further processed to form a colorectal administrative form.

43. The method of claim 42 wherein said colorectal administrative form comprises an enema.

44. The method of any one of claims 2 to 41 wherein said dried powder composition of step (v) is further processed to produce nutraceuticals suitable for oral administration.

45. The method of claim 44, wherein said oral administrative form comprises tablets or capsules.

46. The method of any one of claims 2 to 41 wherein said dried powder composition of step (v) is further processed to produce nutraceuticals suitable for topical administration.

47. The method of claim 46 wherein said topical administrative form comprises topical creams or gels.

48. The method of any one of claims 2 to 41 wherein said dried powder composition of step (v) is mixed with juices, alcoholic beverages and powdered beverages to produce enriched forms of those beverages.

49. The method of claim 48 wherein said dried powder composition of step (v) is mixed with fruit juices.

50. The method of claim 49 wherein said fruit juice comprise guava juice.

51. The method of claim 48 wherein said dried powder composition of step (v) is mixed with beer.

52. The method of claim 48 wherein said dried powder composition of step (v) is mixed with the powder form of milk beverage having chocolate and malt.

53. The method of claim 48 wherein said dried powder composition of step (v) is mixed with coffee powder.

54. The method of any one of claims 2 to 41 wherein said dried powder composition of step (v) is mixed with liquid deodorized cocoa butter.

55. The method of claim 54 wherein said dried powder is mixed with liquid deodorized cocoa butter at a ratio of about 5 to 50% dried powder to about 95 to 50% cocoa butter.

56. The method of claim 54 or 55 wherein said resultant liquid mixture is further processed to form nutraceuticals suitable for oral administration.

57. The method of claim 56 wherein said oral administrative form comprises chewable capsules.

58. The method of claim 54 or 55 wherein said resultant liquid mixture is further processed to form nutraceuticals suitable for colorectal administration.

59. The method of claim 58 wherein said colorectal administrative form comprises a suppository.

60. A nutraceutical composition comprising a combination of oil soluble antioxidants and water soluble antioxidants derived from a plant extract, wherein the plant extract further provides a natural surfactant to the composition.

61. The composition of claim 60 wherein said plant extract is derived from the Vernonia amygdalina plant.

62. The composition of claim 60 or 61 wherein said oil soluble antioxidant is derived from aquatic animal oil.

63. The composition of claim 60 or 61 wherein said oil soluble antioxidant is derived from krill oil or edible fish oil such as cod liver oil or shark liver oil.

64. The composition of claim 60 or 61 wherein said edible oil is vegetable derived oil such as red palm oil, pressed palm fiber oil, wheat germ oil, coconut oil, corn oil, soya bean oil, olive oil, sunflower oil, rice bran oil or grape seed oil.

65. Use of the composition of any one of claims 60 to 64 in the manufacture of a medicament for the prevention of human health complications.

66. The use of claim 65 wherein said health complications comprise cancers of the breast, lung, prostate or colon, cardiovascular disease, fatty liver, asthma, rheumatoid arthritis, dermal allergies, edema, diabetes, hypertension and hyperglycemia.

67. Use of the composition of any one of claims 60 to 64 as an antioxidant, anti-bacterial, anti-viral or anti-fungal agent.

68. Use of the composition of any one of claims 60 to 64 as an immunostimulator.

69. Use of the composition of any one of claims 60 to 64 as a detoxification agent including that suitable for colorectal cleansing.
